(19) United States
(12) Patent Application Publication
(10) Pub. No.: US 2007/0154352 A1
(43) Pub. Date: Jul. 5, 2007

(54) CONCENTRATION DETECTOR AND METHOD OF USING THE SAME

(76) Inventors: Hsi-Ming Shu, Taipei (TW); Feng-Yi Deng, Taipei (TW); Yean-Der Kuan, Taipei (TW); Ya-Chen Chung, Taipei (TW); Yu-Ren Chiou, Taipei (TW)

Correspondence Address: G. LINK CO., LTD., 3550 BELL ROAD, MINOOKA, IL, 60447, US

(21) Appl. No.: 11/617,169

(22) Filed: Dec. 28, 2006

(30) Foreign Application Priority Data
Dec. 30, 2005 (TW) 094147520

(51) Int. Cl. G01N 33/00

(52) U.S. Cl. 422/68.1

(57) ABSTRACT

A concentration detector is disclosed, which is adapted to sense the concentration of a liquid fuel and includes an internal compartment for containing the liquid fuel. The concentration detector comprises a heater, one or more temperature sensors and a concentration calculator. The heater is disposed in the internal compartment of the concentration detector to warm up the liquid fuel. The temperature sensors are disposed in the internal compartment of the concentration detector to measure the temperature of the liquid fuel. The concentration calculator is used to receive the temperature of the liquid fuel measured by the temperature sensors, to calculate a rate of change in the temperature of the liquid fuel, and then to compute a corresponding concentration of the liquid fuel based on the rate of change in the temperature of the liquid fuel.

### FIELD OF THE INVENTION

The present invention relates to a concentration detector, and more particularly, to a sensor for detecting the concentration of liquid fuels in a fuel cell.

### BACKGROUND OF THE INVENTION

A fuel cell is a power generator, which converts chemical energy stored within fuels and oxidants directly into electrical energy through reactions of its electrodes. The types of fuel cells are diverse and their classifications vary. According to the properties of their electrolytes, fuel cells can be divided into five types including alkaline fuel cells, phosphoric acid fuel cells, proton exchange membrane fuel cells, fused carbonate fuel cells, and solid oxide fuel cells. Wherein, a proton exchange membrane fuel cell includes a so-called direct methanol fuel cell (DMFC), which directly uses methanol as fuel without modifying the same into hydrogen gas. This is also at present a technique that can generate relatively high power. Such fuel cells may be applied to large power plants, vehicular power generators, portable power supplies and so forth.

It is essential to control the concentration of liquid fuels while commercializing such types of fuel cells as DMFC. Theoretically, fuels with lower concentrations produce less electricity, and fuels with higher concentrations produce more electricity. Accordingly, to maintain the concentration at a predetermined level, a concentration detector is needed to monitor the concentration of liquid fuels in real-time. As such, the electrical output of fuel cells is regulated, and electronic products using the fuel cells will not be damaged due to unsteady power supplied by the fuel cells.

### SUMMARY OF THE INVENTION

It is a primary object of the invention to provide a concentration detector for a fuel cell, which constantly monitors the concentration of liquid fuels required by a fuel cell, and responds in real-time as the concentration is changed.

In accordance with the aforementioned object of the invention, a concentration detector is provided, which is adapted to sense the concentration of a liquid fuel and includes an internal compartment for containing the liquid fuel. The concentration detector comprises a heater, one or more temperature sensors and a concentration calculator. The heater is disposed in the internal compartment of the concentration detector to warm up the liquid fuel. The temperature sensors are disposed in the internal compartment of the concentration detector to measure the temperature of the liquid fuel. The concentration calculator is used to receive the temperature of the liquid fuel measured by the temperature sensors, to calculate a rate of change in the temperature of the liquid fuel, and then to compute a corresponding concentration of the liquid fuel based on the rate of change in the temperature of the liquid fuel.

### BRIEF DESCRIPTION OF THE DRAWINGS

The foregoing aspects, as well as many of the attendant advantages and features of this invention will become more apparent by reference to the following detailed description, when taken in conjunction with the accompanying drawings, wherein:

FIG. 1A schematically illustrates the structure of a concentration detector according to an embodiment of the invention;

FIG. 1B is plot showing the relationship between the temperature of the liquid fuel and time for heating the same in FIG. 1A;

FIG. 2 schematically illustrates a concentration detector according to an embodiment of the invention; and

FIG. 3 is a flow chart of the process of detecting the concentration of liquid fuels according to an embodiment of the invention.

### DETAILED DESCRIPTION OF THE INVENTION

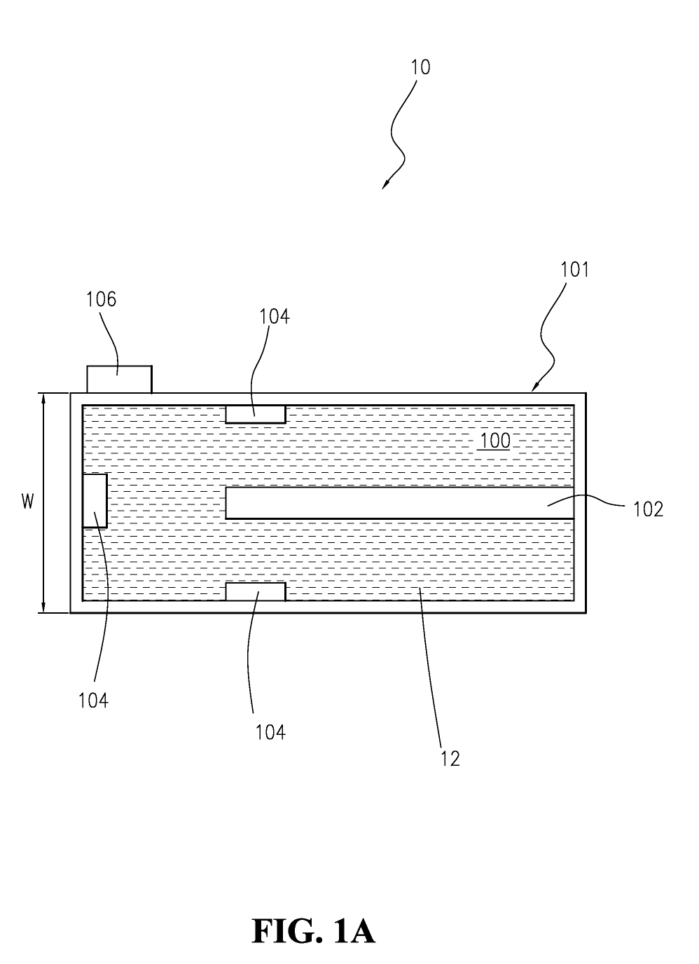

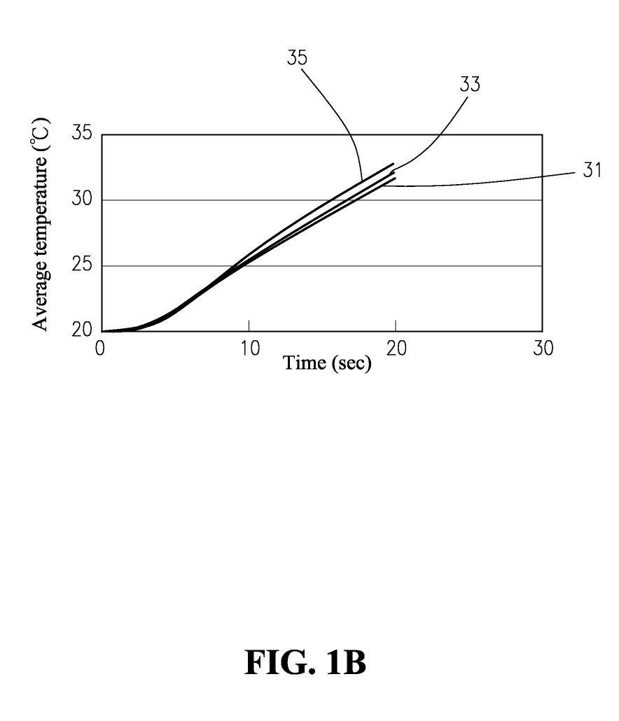

FIG. 1A schematically illustrates the structure of a concentration detector according to an embodiment of the invention. A concentration detector 10 is adapted to sense the concentration of the liquid fuel 12, and includes an internal compartment 100 for containing the liquid fuel 12. The liquid fuel 12 may be a solution of methanol. The internal compartment 100 may be a small space with a width W of 1 millimeter (mm) to 3 mm. As shown in FIG. 1A, the concentration detector 10 further includes a hollow case 101. The hollow case 101 may be made of heat-insulating material, or heat-insulating material may cover the hollow case 101, so as to keep the heat capacity within the internal compartment 100, prevent heat from radiating and reduce the effect of the surroundings on the temperature of the liquid fuel 12 inside the internal compartment 100.

Referring to FIG. 1A, the concentration detector 10 includes a heater 102, one or more temperature sensors 104 and a concentration calculator 106, which are separately described hereinafter.

The heater 102 is disposed in the internal compartment 100 of the concentration detector 10 for warming up the liquid fuel 12. The heater 102 is a heating bar or a heating wire, for example. The heater 102 may supply regular power to heat the liquid fuel 12. Additionally, the surface of the heater 102 may treated by an anticorrosive process and/or an acid-proof process.

The temperature sensor 104 is positioned in the internal compartment 100 of the concentration detector 10 for measuring the temperature of the liquid fuel 12 at different locations of the internal compartment 100. Furthermore, the surface of the temperature sensor 104 may treated by an anticorrosive process and/or an acid-proof process.

The concentration calculator 106 receives the data of the temperature of the liquid fuel 12 measured by the temperature sensors 104, calculates a rate of change in the temperature of the liquid fuel 12, and then computes a corresponding concentration of the liquid fuel based on the rate of change in the temperature of the liquid fuel 12. The concentration calculator 106 may include a microprocessor. The concentration calculator 106 may be electrically coupled to the temperature sensors 104. Also, the heater 102 may be electrically connected to the concentration calculator 106. Thereby, the concentration calculator 106 controls the heater 102 and controls the heating power and duration for the heater 102.

FIG. 1B shows the relationship between the temperature of the liquid fuel 12 and the time for heating the same in FIG. 1A. For example, the heater 102 continuously heats the liquid fuel 12 with a power of 0.5 watts. With reference to FIG. 1B, the curves 31, 33, and 35 represent the relationship between the average temperature of the liquid fuel 12 measured by the temperature sensors 104 and the heating time 102, which result from three liquid fuels with concentrations of 5%, 10% and 20%, respectively. It should be noted that the slopes of the curves 31, 33, and 35 (also referred to as the rate of change in temperature) are different. Hence, the corresponding concentration of liquid fuel can be estimated from the rate of change in the temperature of the liquid fuel 12.

Assuming the average temperature of the liquid fuel 12 measured by the temperature sensors 104 is 20° C., then the concentration calculator 106 will receive the temperature datum (i.e. 20° C.) and compute a temperature change rate of 0.7° C./sec. Thereafter, the concentration calculator 106 matches the rate of 0.7° C./sec with the slope of a curve depicting the concentration of 20% according to the experimental database as illustrated in FIG. 1B. As such, the concentration detector 10 uses the rate of 0.7° C./sec to determine that the current concentration of the liquid fuel 12 is 20%.

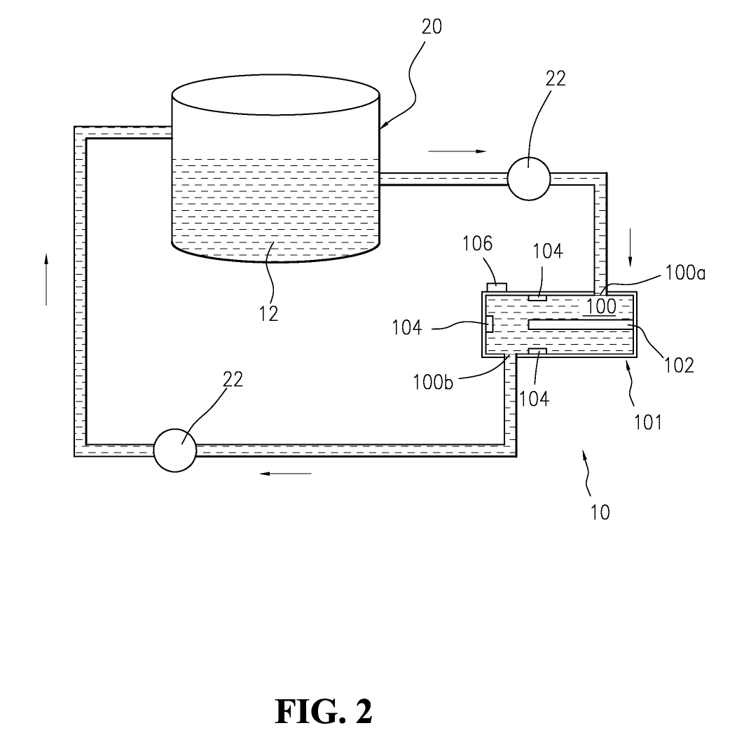

FIG. 2 schematically illustrates a concentration detector 10 according to an embodiment of the invention. Referring to FIG. 2, the concentration detector 10 is used to detect the concentration of the liquid fuel 12 inside a fuel supply tank 20. The fuel supply tank 20 provides fuels for a fuel cell. The concentration detector 10 may be equipped with an inlet 100a to receive the liquid fuel from the fuel supply tank 20, as well as an outlet 100b to deliver the liquid fuel within the internal compartment 100 back to the fuel supply tank 20.

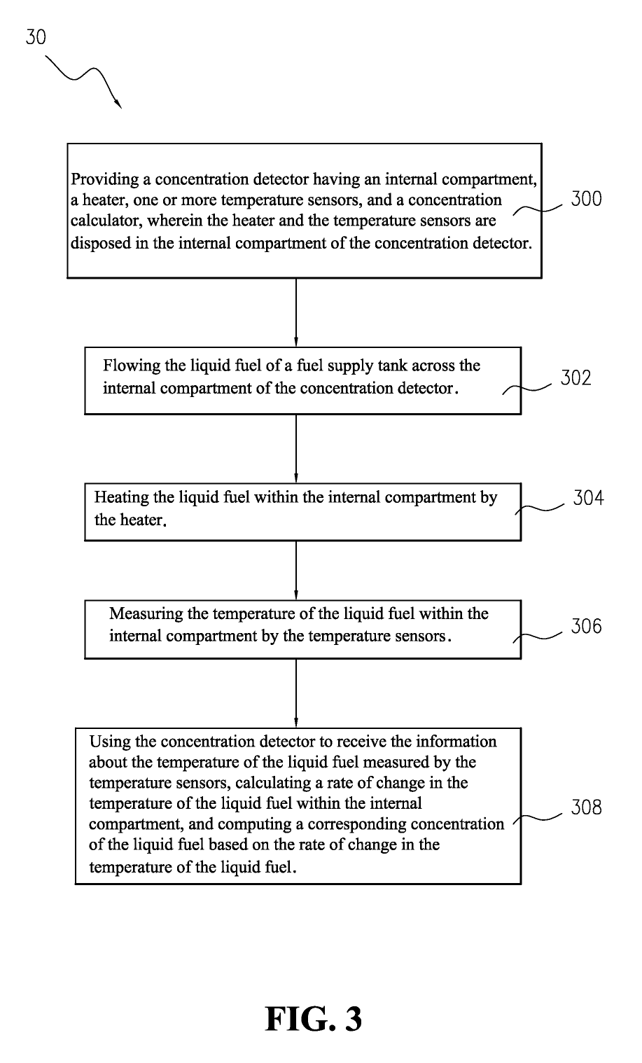

FIG. 3 is a flow chart of the process of detecting the concentration of liquid fuels according to an embodiment of the invention. A method 30 is performed to sense the concentration of the liquid fuel 12 inside a fuel supply tank 20. The fuel supply tank 20 provides fuels, such as a methanol solution, for a fuel cell. The method 30 includes steps 300 through 308, which are separately described hereinafter in conjunction with the embodiment in FIG. 2.

In step 300, a concentration detector 10 is provided. The concentration detector 10 includes an internal compartment 100, a heater 102, one or more temperature sensors 104, and a concentration calculator 106. The heater 102 and the temperature sensors 104 are disposed in the internal compartment 100 of the concentration detector 10.

Step 302 is performed to flow the liquid fuel 12 inside the fuel supply tank 20 across the internal compartment 100 of the concentration detector 10. In step 304, the heater 102 warms up the liquid fuel 12 within the internal compartment 100 at a constant power. While being heated, the liquid fuel 12 within the internal compartment 100 is still such that the heat capacity is completely absorbed by the liquid fuel 12 in the internal compartment 100 without dispersing due to the flow of the liquid fuel.

In step 306, one or more temperature sensors 104 measure the temperature of the liquid fuel 12 within the internal compartment 100. In step 308, the concentration calculator 106 receives the information about the temperature of the liquid fuel 12 measured by the temperature sensors 104, calculates a rate of change in the temperature of the liquid fuel 12 in the internal compartment 100, and then computes the concentration of the liquid fuel corresponding to the rate of change in the temperature of the liquid fuel 12.

The method 30 further includes constructing a transporting loop for circulating the liquid fuel 12 between the fuel supply tank 20 and the concentration detector 10 as indicated by the arrows in FIG. 2. One or more pumps 22 may be positioned at the transporting loop to propel the liquid fuel 12 flow. The concentration detector 10 may be equipped with an inlet 100a for receiving the liquid fuel from the fuel supply tank 20, and an outlet 100b for delivering the liquid fuel in the internal compartment 100 back to the fuel supply tank 20.

In order to measure the temperature of the liquid fuel 12 within the internal compartment 100 more accurately, the method 30 also includes calculating an average of the temperature of the liquid fuel 12 measured by the temperature sensors 104. The average stands for the current temperature of the liquid fuel 12.

To sum up, the invention possesses the following features and efficacies, wherein:
1. Because the structure of the concentration detector is not complicated in accordance with the invention, mass production of such concentration detector is easy and costs less; and

2. It is convenient to identify the concentration of the liquid fuel since the concentration detector of the invention is sensitive. Moreover, the varied concentration of the liquid fuel can be monitored in real-time by such a concentration detector.

While the invention has been particularly shown and described with reference to the preferred embodiments thereof, these are, of course, merely examples to help clarify the invention and are not intended to limit the invention. It will be understood by those skilled in the art that various changes, modifications, and alterations in form and details may be made therein without departing from the spirit and scope of the invention, as set forth in the following claims.

What is claimed is:

1. A concentration detector adapted to sense a concentration of a liquid fuel, having an internal compartment for containing the liquid fuel, the concentration detector comprising:
a heater disposed in the internal compartment of the concentration detector to warm up the liquid fuel;
one or more temperature sensors disposed in the internal compartment of the concentration detector to measure the temperature of the liquid fuel; and
a concentration calculator used to receive the temperature of the liquid fuel measured by the temperature sensors, to calculate a rate of change in the temperature of the liquid fuel, and then to compute a corresponding concentration of the liquid fuel based on the rate of change in the temperature of the liquid fuel.

2. The concentration detector of claim 1, wherein the heater applies a constant power for heating.

3. The concentration detector of claim 1, wherein the concentration detector comprises an inlet for receiving a liquid fuel output by a fuel supply tank, and an outlet for delivering a liquid fuel within the internal compartment back to the fuel supply tank.

4. The concentration detector of claim 1, wherein the fuel supply tank provides fuels for a fuel cell.

5. The concentration detector of claim 1, wherein the liquid fuel is a solution of methanol.

6. The concentration detector of claim 1, wherein the width of the internal compartment ranges between 1 millimeter and 3 millimeters.

7. The concentration detector of claim 1, wherein the heater is a heating bar and/or a heating wire.

8. The concentration detector of claim 1, wherein a surface of the heater is treated by an anticorrosive process and/or an acid-proof process.

9. The concentration detector of claim 1, wherein a surface of the temperature sensor is treated by an anticorrosive process and/or an acid-proof process.

10. The concentration detector of claim 1, wherein the concentration calculator comprises a microprocessor.

11. The concentration detector of claim 2, wherein the power for heating is 0.5 watts.

12. The concentration detector of claim 1, wherein the concentration detector comprises a hollow case made of a heat-insulating material.

13. A method of detecting a concentration of a liquid fuel, which is used to sense a concentration of a liquid fuel within a fuel supply tank, where the fuel supply tank provides fuels for a fuel cell, the method comprising:
providing a concentration detector, wherein the concentration detector comprises an internal compartment, a heater, one or more temperature sensors, and a concentration calculator, and the heater and the temperature sensors are disposed in the internal compartment of the concentration detector;
flowing a liquid fuel output by the fuel supply tank across the internal compartment of the concentration detector;
using the heater to warm up a liquid fuel within the internal compartment;
using the temperature sensors to measure the temperature of the liquid fuel within the internal compartment; and
using the concentration detector to receive the temperature of the liquid fuel measured by the temperature sensors, to calculate a rate of change in the temperature of the liquid fuel within the internal compartment, and to compute a corresponding concentration of the liquid fuel based on the rate of change in the temperature of the liquid fuel.

14. The method of claim 13, further comprising constructing a transporting loop between the fuel supply tank and the concentration detector to circulate the liquid fuel.

15. The method of claim 13, further comprising calculating an average of the temperature of the liquid fuel measured by the temperature sensors, wherein the average stands for a current temperature of the liquid fuel.

16. The method of claim 14, wherein the concentration detector comprises an inlet for receiving a liquid fuel from the fuel supply tank, and an outlet for delivering a liquid fuel within the internal compartment back to the fuel supply tank.

17. The method of claim 13, wherein the liquid fuel is a solution of methanol.

18. The method of claim 13, wherein the heater applies a constant power for heating.
